(19) United States
(12) Patent Application Publication
(10) Pub. No.: US 2015/0358740 A1
(43) Pub. Date: Dec. 10, 2015

(54) ELECTRICAL TUNING OF PARAMETERS OF PIEZOELECTRIC ACTUATED TRANSDUCERS

(71) Applicant: INVENSENSE, INC., San Jose, CA (US)

(72) Inventors: Julius Ming-Lin Tsai, San Jose, CA (US); Stephen Lloyd, Los Altos, CA (US)

(21) Appl. No.: 14/295,881

(22) Filed: Jun. 4, 2014

(51) Int. Cl. H04R 17/10; A61B 5/117; G01N 29/36; H04R 3/04

(52) U.S. Cl. CPC H04R 17/10; H04R 3/04; A61B 5/1172; G01N 29/36

(57) ABSTRACT

Parameters, such as, quality factor and/or resonance frequency of an acoustic transducer can be electrically tuned. The acoustic transducer can include a piezoelectric layer deposited on a silicon supporting layer, a first electrode layer deposited on the piezoelectric layer, and a second electrode layer deposited between the silicon supporting layer and piezoelectric layer. In one aspect, a resonant frequency of the piezoelectric actuated transducer is electrically tuned based on modifying a voltage across at least a portion of the first electrode layer and the second electrode layer. In another aspect, a quality factor of the piezoelectric actuated transducer is electrically tuned based on modifying a resistance across at least another portion of the first electrode layer and the second electrode layer.

### TECHNICAL FIELD

The subject disclosure relates to piezoelectric actuated transducers, e.g., to electrically tuning parameters of piezoelectric actuated transducers.

### BACKGROUND

Piezoelectric materials facilitate conversion between mechanical energy and electrical energy. Moreover, a piezoelectric material can generate an electrical signal when subjected to mechanical stress, and can vibrate when subjected to an electrical voltage. Piezoelectric materials are widely utilized in piezoelectric ultrasonic transducers to generate acoustic waves based on an actuation voltage applied to electrodes of the piezoelectric ultrasonic transducer. Generally, the piezoelectric ultrasonic transducer comprises a membrane with the piezoelectric material, a supporting layer, and electrodes combined with a cavity beneath the electrodes. Typically, the cavity comprises vacuum and therefore damping contributed from the cavity can be ignored. However, acoustic media (e.g., air, water, liquid) on the other side of the membrane can significantly change the damping of the transducer. As an example, a quality factor of over 20 can be observed when the piezoelectric ultrasonic transducer is operating in air, but can drop down to lower than 2 if the piezoelectric ultrasonic transducer is submerged in water.

Further, residual stress on the membrane can cause a drift in a resonance frequency of the piezoelectric material. Typically, the residual stress distribution over the piezoelectric material is not uniform, resulting in a non-uniform piezoelectric ultrasonic transducer. Both the residual stress and the quality factor cause a mismatch between individual piezoelectric ultrasonic transducers in an array and can substantially decrease overall wafer yield.

### SUMMARY

The following presents a simplified summary of the specification to provide a basic understanding of some aspects of the specification. This summary is not an extensive overview of the specification. It is intended to neither identify key or critical elements of the specification nor delineate any scope particular to any embodiments of the specification, or any scope of the claims. Its sole purpose is to present some concepts of the specification in a simplified form as a prelude to the more detailed description that is presented later.

The systems and methods described herein, in one or more embodiments thereof, relate to a piezoelectric actuated transducer that comprises a piezoelectric layer that is deposited on a silicon supporting layer, a first electrode layer that is deposited on the piezoelectric layer, and a second electrode layer that is deposited between the silicon supporting layer and piezoelectric layer. In one aspect, at least a portion of the first electrode layer and the second electrode layer is coupled to a direct current (DC) voltage source to facilitate electrical tuning of a resonant frequency of the piezoelectric actuated transducer. In another aspect, at least a portion of the first electrode layer and the second electrode layer is coupled to a resistor to facilitate electrical tuning of a quality factor of the piezoelectric actuated transducer.

Another aspect of the disclosed subject matter relates to a method that comprises depositing a piezoelectric layer on a silicon supporting layer and forming a first electrode layer on the piezoelectric layer and a second electrode layer between the silicon supporting layer and piezoelectric layer to form a piezoelectric actuated transducer. Further, the method comprises tuning a resonant frequency of the piezoelectric actuated transducer based on controlling a voltage signal across at least a portion of the first electrode layer and the second electrode layer. Furthermore, the method comprises tuning a quality factor of the piezoelectric actuated transducer based on controlling a resistance across at least another portion of the first electrode layer and the second electrode layer.

Yet another aspect of the disclosed subject matter relates to a biometric sensing method that comprises transmitting an ultrasonic signal by an acoustic sensing element that has a voltage source coupled across a first electrode deposited on a piezoelectric layer and a second electrode deposited below the piezoelectric layer. Further, the method comprises sensing, by the piezoelectric layer, an interference signal that is generated based on an interference of the ultrasonic signal with an object. Furthermore, the method comprises controlling a voltage of the voltage source to tune a resonant frequency of the piezoelectric layer

The following description and the annexed drawings set forth certain illustrative aspects of the specification. These aspects are indicative, however, of but a few of the various ways in which the principles of the specification may be employed. Other advantages and novel features of the specification will become apparent from the following detailed description of the specification when considered in conjunction with the drawings.

### BRIEF DESCRIPTION OF THE DRAWINGS

The numerous aspects, embodiments, objects and advantages of the present invention will be apparent upon consideration of the following detailed description, taken in conjunction with the accompanying drawings, in which like reference characters refer to like parts throughout, and in which:

FIG. 1 illustrates an example tuning system that can be utilized to electrically control parameters of an acoustic transducer;

FIG. 2 illustrates an example system for acoustic sensing;

FIG. 3 illustrates an example system utilized for electrical tuning of a quality factor of an acoustic transducer;

FIG. 4 illustrates an example system utilized for electrical tuning of a resonance frequency of an acoustic transducer;

FIG. 5 illustrates an example system that facilitates electrically tuning both the quality factor and resonant frequency of the acoustic transducer;

FIGS. 6A-B illustrate simulation results for electrical tuning of parameters of the acoustic transducer;

FIG. 7 illustrates an example system utilized for analysis of acoustically sensed data;

FIG. 8 illustrates an example methodology for controlling a quality factor of an acoustic sensor; and

FIG. 9 illustrates an example methodology for controlling a resonance frequency of an acoustic sensor.

### DETAILED DESCRIPTION OF THE INVENTION

One or more embodiments are now described with reference to the drawings, wherein like reference numerals are used to refer to like elements throughout. In the following description, for purposes of explanation, numerous specific details are set forth in order to provide a thorough understanding of the various embodiments. It may be evident, however, that the various embodiments can be practiced without these specific details, e.g., without applying to any particular networked environment or standard. In other instances, well-known structures and devices are shown in block diagram form in order to facilitate describing the embodiments in additional detail.

Systems and methods disclosed herein, in one or more aspects provide electrical tuning of a quality factor and/or resonance frequency of an acoustic transducer (e.g., a piezoelectric actuated transducer). The subject matter is described with reference to the drawings, wherein like reference numerals are used to refer to like elements throughout. In the following description, for purposes of explanation, numerous specific details are set forth in order to provide a thorough understanding of the subject innovation. However, that the subject matter may be practiced without these specific details.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form. In addition, the word “coupled” is used herein to mean direct or indirect electrical or mechanical coupling. In addition, the words “example” and “exemplary” are used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

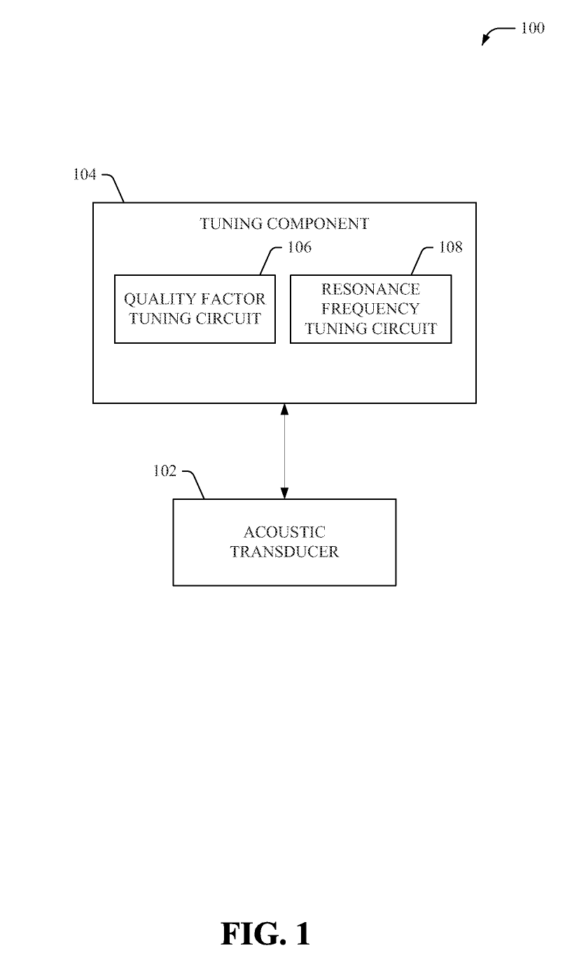

Initially, referring to FIG. 1, there illustrated is an example tuning system 100 that can be utilized to electrically control parameters of an acoustic transducer 102. The acoustic transducer 102 can include most any electrical device that converts an audio signal (e.g., sound waves) into mechanical vibrations and/or electrical signals. In one aspect, the acoustic transducer 102 can comprise an acoustic sensing element (e.g., a piezoelectric element) that generates and senses ultrasonic sound waves. An object in a path of the generated sound waves can create an interference (e.g., changes in frequency or phase, reflection signal, echoes, etc.) that can then be sensed by the acoustic sensing element. The interference can be analyzed to determine physical parameters such as (but not limited to) distance, density and/or speed of the object. As an example, the acoustic transducer 102 can be utilized in various applications, such as, but not limited to, wireless devices, industrial systems, automotive systems, robotics, telecommunications, security, medical devices, etc. Typically, the acoustic transducer 102 can be part of a sensor array comprising a plurality of acoustic transducers deposited on a wafer.

According to an embodiment, a tuning component 104 can be utilized to control and/or tune parameters, such as, a quality factor and/or a resonance frequency of the acoustic transducer 102. The tuning component 104 can comprise one or more electronic circuits that can be utilized to modify the quality factor and/or the resonance frequency. As an example, a quality factor tuning circuit 106 can be utilized to facilitate tuning of the quality factor. Moreover, the quality factor of the acoustic transducer 102 can change based on damping contributed by material, such as, an acoustic medium (e.g., air, liquid, human tissue, etc.) at (or near) the surface of acoustic transducer 102 or that the acoustic transducer 102 is placed within. In one aspect, the quality factor tuning circuit 106 can provide a path for dissipation of energy stored in the acoustic transducer 102 due to the damping. As an example, the quality factor tuning circuit 106 can include a resistor that facilitates the energy dissipation.

In another aspect, the tuning component 104 can comprise a resonance frequency tuning circuit 108 that can be utilized to facilitate tuning of the resonance frequency of the acoustic transducer 102. Moreover, residual stress of the acoustic sensing element causes a non-uniform residual stress distribution across the wafer leading to a resonance frequency drift. In one aspect, the resonance frequency tuning circuit 108 can generate tensile or compressive stress in the acoustic sensing element. For example, the resonance frequency tuning circuit 108 can include a direct current (DC) voltage source that facilitates stress generation to compensate for the resonance frequency drift. Both residual stress and quality factor have a substantial impact on the uniformity of the acoustic transducer 102 and can cause a mismatch between individual acoustic transducers in an array on a wafer. Accordingly, the tuning component 104 can be utilized to electrically tune the resonance frequency and/or quality factor to reduce the mismatch and improve the overall wafer yield.

It is noted that the design of system 100 can include different component selections, topologies, etc., to achieve electrical tuning of the resonance frequency and/or quality factor. Moreover, it is noted that the acoustic transducer 102, tuning component 104, the quality factor tuning circuit 106, and the resonance frequency tuning circuit 108 can include most any electrical circuit(s) that can include components and circuitry elements of any suitable value in order to implement the embodiments of the subject innovation. Furthermore, it can be appreciated that the components of system 100 can be implemented on one or more integrated circuit (IC) chips.

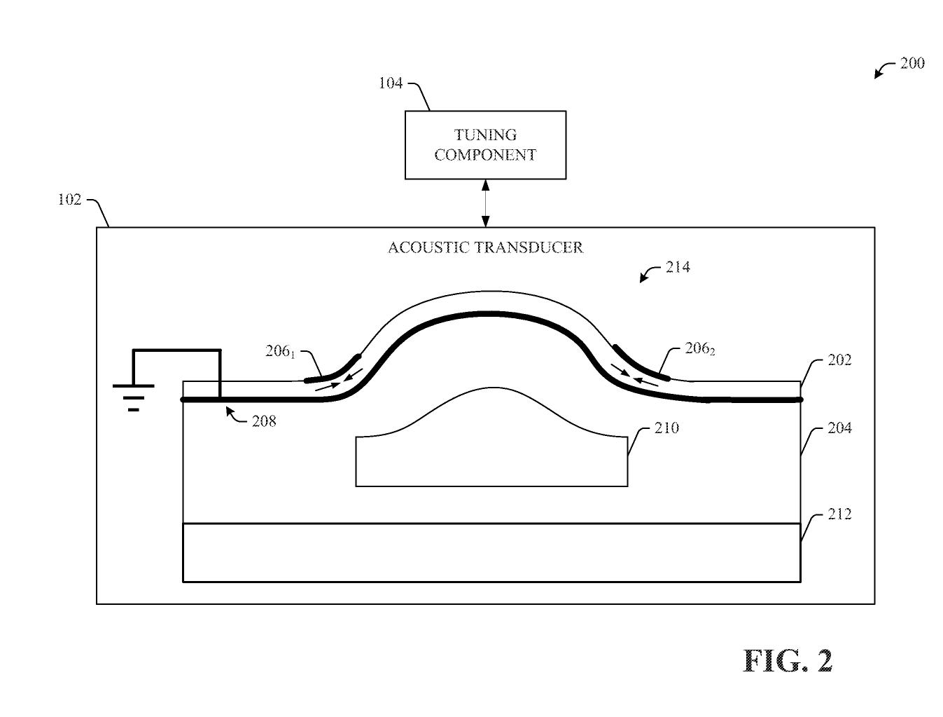

Referring now to FIG. 2, there illustrated is an example system 200 for acoustic sensing, according to an aspect of the specification. The acoustic transducer 102 and the tuning component 104 can operate and include functionality, as more fully described with respect to system 100. In an aspect, the acoustic transducer 102 can comprise a piezoelectric ultrasonic transducer that employs a piezoelectric material 202, such as, but not limited to, Aluminum nitride (AlN), to facilitate acoustic sensing. Specifically, the piezoelectric material 202 can generate electric charges under mechanical stress and conversely experience a mechanical strain in the presence of an electric field. More specifically, the piezoelectric material 202 can sense mechanical vibrations caused by an acoustic signal and produce an electrical charge at the frequency (e.g., ultrasonic frequency) of the vibrations. Additionally, the piezoelectric material 202 can generate an acoustic wave by vibrating in an oscillatory fashion at the same frequency (e.g., ultrasonic frequency) as an input current generated by an alternating current (AC) voltage applied across the piezoelectric material 202. It is noted that the piezoelectric material 202 can include most any material (or combination of materials) that exhibits piezoelectric properties, such that the structure of the material does not have a center of symmetry and a tensile or compressive stress applied to the material alters the separation between positive and negative charge sights in a cell causing a polarization at the surface of the material. The polarization is directly proportional to the applied stress and is direction dependent so that compressive and tensile stresses results in electric fields of opposite voltages.

According to an aspect, the acoustic transducer 102 comprises a membrane (e.g., a microelectromechanical systems (MEMS) membrane) with the piezoelectric material 202, with (or without) a supporting layer 204 and actuating electrodes (206₁-206₂ and 208) combined with a cavity 210 beneath. As an example, a released structure, in plane with the membrane, can include a silicon dioxide (SiO₂) layer that can be utilized as the supporting layer 204. Further, MN can be utilized as the piezoelectric material 202 with top (206₁-206₂) and bottom (208) metal layers (e.g., Aluminum (Al)/Titanium (Ti), Molybdenum (Mo), etc.) patterned to form electrodes in particular shapes (e.g., circle, square, octagon, hexagon, etc.) that are defined in-plane with the membrane. Furthermore, in one example, one of the top electrodes (206₁-206₂) can be placed at a maximum strain area of the membrane and another can be placed at close to a constraint (e.g., a flexible structure in connection to an anchor of the membrane). It is noted that the bottom electrode 208 can also be formed by a supporting silicon layer with heavy doping (e.g., having a doping level greater than a defined threshold). The cavity 210 which allows the membrane to move is formed within the supporting layer 204.

When actuation voltage is applied to the electrodes (206₁-206₂ and 208), the acoustic transducer 102 will deform and move out of plane with respect to the surface of the substrate 212. The motion then pushes the acoustic media 214 it is in contact with and an acoustic wave is generated. Oftentimes, vacuum is present inside the cavity 210 and therefore damping contributed from the media within the cavity 210 can be ignored. However the acoustic media 214 on the other side of the membrane can substantially change the damping of the acoustic transducer 102. For example, a quality factor greater than 20 can be observed when the acoustic transducer 102 is operating in air with atmosphere pressure (e.g., acoustic media 214 is air) and can decrease lower than 2 if the acoustic transducer 102 is operating in water (e.g., acoustic media 214 is water). In one aspect, the tuning component 104 can be utilized to compensate for the changes in the quality factor. Moreover, the tuning component 104 (e.g., via the quality factor tuning circuit 106) can provide a path for dissipation of energy stored in the acoustic transducer 102 due to the damping.

Another impact on the acoustic transducer 102 is the residual stress of the piezoelectric material 202, which can cause non-uniform residual stress distribution. As an example, a hill-like contour can be observed for distribution of stress in the piezoelectric material 202 layer, which can cause a mismatch between individual acoustic transducers in an array. In an aspect, the tuning component 104 can be utilized to compensate for the non-uniform residual stress distribution. Moreover, the tuning component 104 (e.g., via the resonance frequency tuning circuit 108) can generate tensile or compressive stress in the acoustic transducer 102 to balance the non-uniform residual stress distribution.

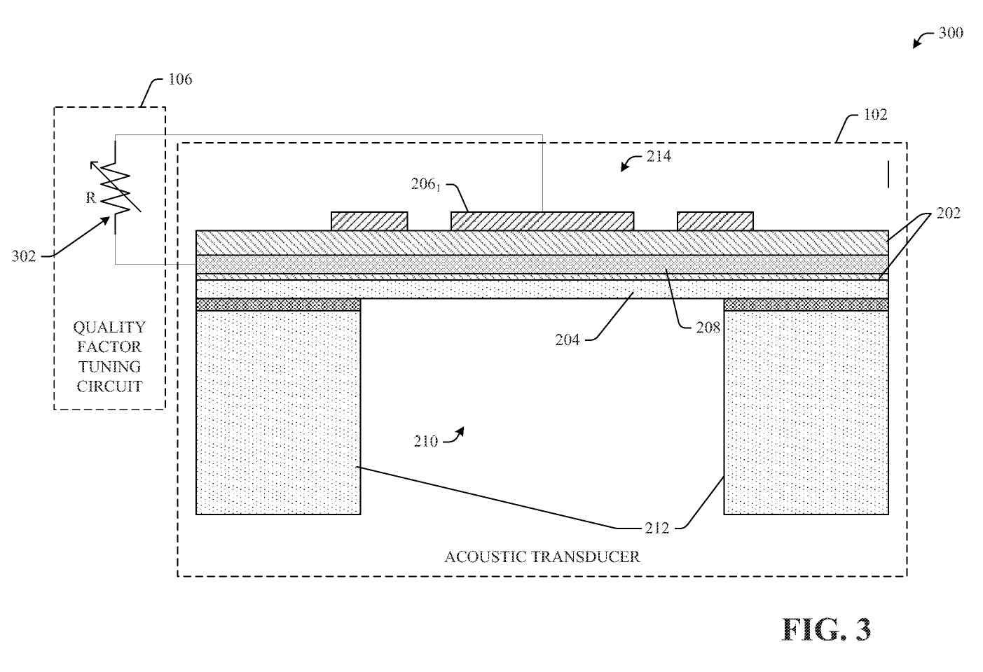

FIG. 3 illustrates an example system 300 utilized for electrical tuning of a quality factor of an acoustic transducer 102, according to an aspect of the specification. A quality factor comprises a parameter that represents damping of an transducer; a higher quality factor represents a slower rate of energy loss relative to the stored energy of the transducer (e.g., the oscillations can fade at a slower rate), whereas a lower quality factor represents a faster rate of energy loss relative to the stored energy of the transducer (e.g., the oscillations can fade at a faster rate). As an example, the quality factor of the acoustic transducer 102 can be determined based on a ratio of the energy stored in the acoustic transducer 102 to the energy dissipated per cycle. Moreover, the quality factor also represents central frequency divided by the bandwidth (e.g., a range of frequencies generated by the acoustic transducer 102). It is noted that the acoustic transducer 102, the quality factor tuning circuit 106, piezoelectric material 202, the supporting layer 204, the electrodes 206₁ and 208, the cavity 210, the substrate 212, and the acoustic media 214 can operate and/or include functionality, as more fully described with respect to systems 100-200.

In one example, acoustic transducer 102 can be (but is not limited to) a piezoelectric micromachined ultrasonic transducer (pMUT) structure manufactured on a silicon-on-insulator (SOI) substrate 212, the piezoelectric material 202 and metal layers (206₁ and 208) are disposed on the substrate 212. The metal layers are patterned to form the electrodes (206₁ and 208). According to an aspect, the electrode 206₁ can be located at an area of the piezoelectric material 202 having maximum strain/deformation (e.g., or strain greater than a defined strain threshold). Collection of electrode charge is based on how much information can be sensed, so placing the electrode 206₁ at the maximum strain area can enable collection of a maximum amount of charges that can be utilized to maximize the quality factor. As an example, for a circular membrane the electrode 206₁ can be located at (or near, or within a defined distance from) the center of the circular membrane.

In one aspect, a set of the electrodes (206₁ and 208) are coupled to a quality factor tuning circuit 106 to control the quality factor. According to an embodiment, the quality factor tuning circuit 106 comprises a resistor with resistance R (ohms) that is coupled across the electrodes (206₁ and 208). As an example, the resistance R of the resistor 302 can be controlled by most any control circuit (not shown) to modify the quality factor of the acoustic transducer 102. It is noted that the resistor 302 can have a suitable resistance values R depending on the application of the acoustic transducer 102. For instance, resistor 302 can have a variable resistance ranging from 10 kΩ to 1MΩ. Moreover, the resistor 302 can be programmable (e.g., resistance value can be changed based on defined instructions/policies, events, etc.) and/or time variant (e.g., resistance value can be changed based on time). As an example, a flexibility of the acoustic transducer 102 can be increased by enabling the acoustic transducer 102 to operate in different modes during different time periods. In an example first mode if the quality factor is to be maximized, the quality factor tuning can be relaxed, whereas in an example second mode, if the quality factor is to be reduced then the damping can be reduced by modifying the resistance R.

According to an aspect, the resistor 302 provides an energy dissipation path alternative to mechanical damping. As the acoustic transducer 102 deforms and some energy is converted into electrical charges, the resistor 302 can direct the electrical charges outside of the acoustic transducer 102 and facilitate conversion of the electrical charges into another form of energy, for example, heat for dissipation. Accordingly, the quality factor of the acoustic transducer 102 can be electrically tuned. In one example, the value R of resistance of the resistor 302 is structure dependent and can be determined based on structural parameters associated with the acoustic transducer 102, such as, but not limited to membrane diameter, pressure load, etc.

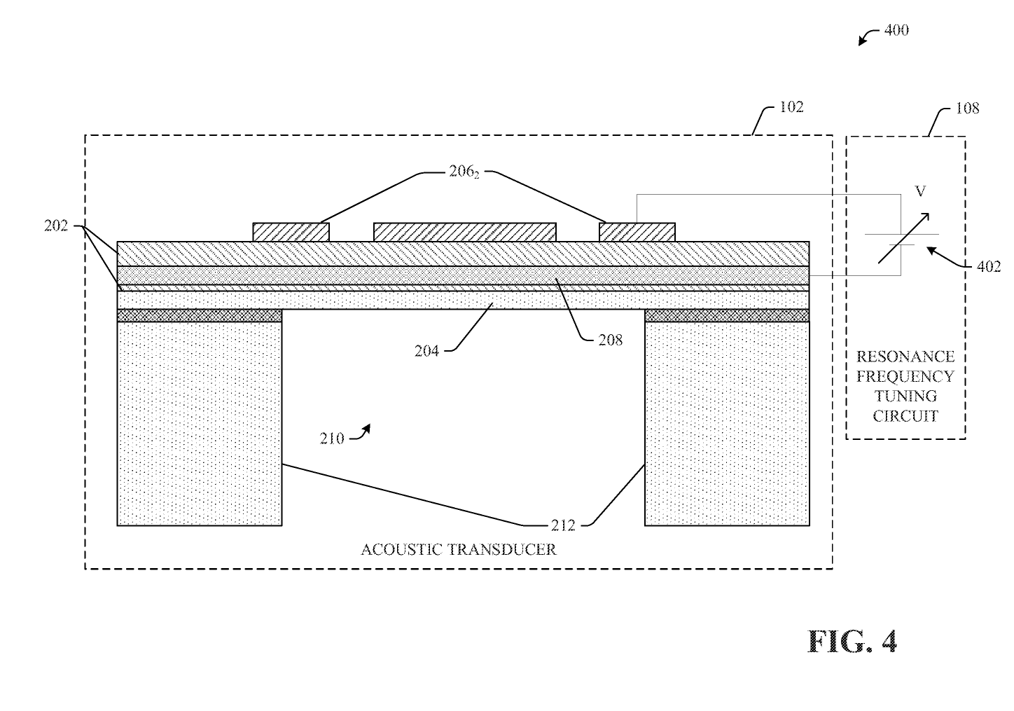

FIG. 4 illustrates an example system 400 utilized for electrical tuning of a resonance frequency of an acoustic transducer 102 in accordance with an aspect of the disclosure. The resonance frequency is a frequency at which the acoustic transducer 102 is most efficient in converting electrical energy to acoustic energy and vice versa. As an example, the resonance frequency can be based on the thickness of the piezoelectric material 202; generally, a thicker layer of piezoelectric material 202 will have a higher resonance frequency than a thinner layer of the piezoelectric material 202 (of the same shape). Moreover, the resonance frequency can be a natural frequency of vibration of the piezoelectric material 202, wherein the acoustic transducer 102 absorbs more energy when it is forced or driven at a frequency that matches the natural frequency than it does at other frequencies. Further, the resonance frequency can be determined based on a spring constant and the mass of the piezoelectric material 202. It is noted that the acoustic transducer 102, the quality factor tuning circuit 106, the piezoelectric material 202, the supporting layer 204, the electrodes 206₂ and 208, the cavity 210, the substrate 212, and the acoustic media 214 can operate and/or include functionality, as more fully described with respect to systems 100-300.

In one example, the acoustic transducer 102 can be (but is not limited to) a pMUT structure manufactured on a silicon-on-insulator (SOI) substrate 212, the piezoelectric material 202 and metal layers (206₂ and 208) are disposed on the substrate 212. The metal layers are patterned to form the electrodes (206₂ and 208). According to an aspect, the electrode 206₂ can be located at an area close to (e.g., within a defined distance from) a constraint of the piezoelectric material 202, for example, a point at which the membrane can be easily strengthened. As an example, the constraint can be a flexible structure in connection to an anchor of the membrane. Typically, for a circular membrane the electrode 206₂ can be located at (or near, or within a defined distance from) a periphery (e.g., circumference) of the circular membrane.

In one aspect, a set of the electrodes (206₂ and 208) are coupled to a resonance frequency tuning circuit 108 to control the resonance frequency of the acoustic transducer 102. According to an embodiment, the resonance frequency tuning circuit 108 comprises a DC voltage source 402 with bias voltage V (volts) that is coupled across the electrodes (206₂ and 208). As an example, the bias voltage V of the DC voltage source 402 can be controlled by most any control circuit (not shown) to modify the resonance frequency. It is noted that the bias voltage V of the voltage source 402 can be modified depending on the application of the acoustic transducer 102. For instance, the voltage source 402 can have a variable bias voltage ranging from 0V-40V. Moreover, the voltage source 402 can be programmable (e.g., bias voltage value can be changed based on defined instructions/policies, events, etc.) and/or time variant (e.g., bias voltage value can be changed based on time). As an example, a flexibility of the acoustic transducer 102 can be increased by enabling the acoustic transducer 102 to operate in different modes during different time periods. During an example first mode, if the resonance frequency is to be increased, the bias voltage can be reduced, whereas during an example second mode, if the resonance frequency is to be reduced, then the bias voltage can be increased. For example, during a frequency modulation mode, different frequencies can be employed by the acoustic transducer 102 at different times. Accordingly, the bias voltage V can be programmed to change to correspond to the different frequencies at the different times and thus, the acoustic transducer 102 can operate at different resonance frequencies at different times, for example, to generate a frequency modulated signal.

According to an aspect, the voltage source 402 introduces tensile or compressive stress in the piezoelectric material 202. The stress will stiffen or soften the equivalent spring constant of the piezoelectric material 202. As the equivalent spring constant changes, the resonance frequency is modified. Accordingly, the resonance frequency of the acoustic transducer 102 can be electrically tuned. In one example, the value V of voltage of the voltage source 402 of is structure dependent and can be determined based on structural parameters associated with the acoustic transducer 102, such as, but not limited to membrane diameter, pressure load, etc.

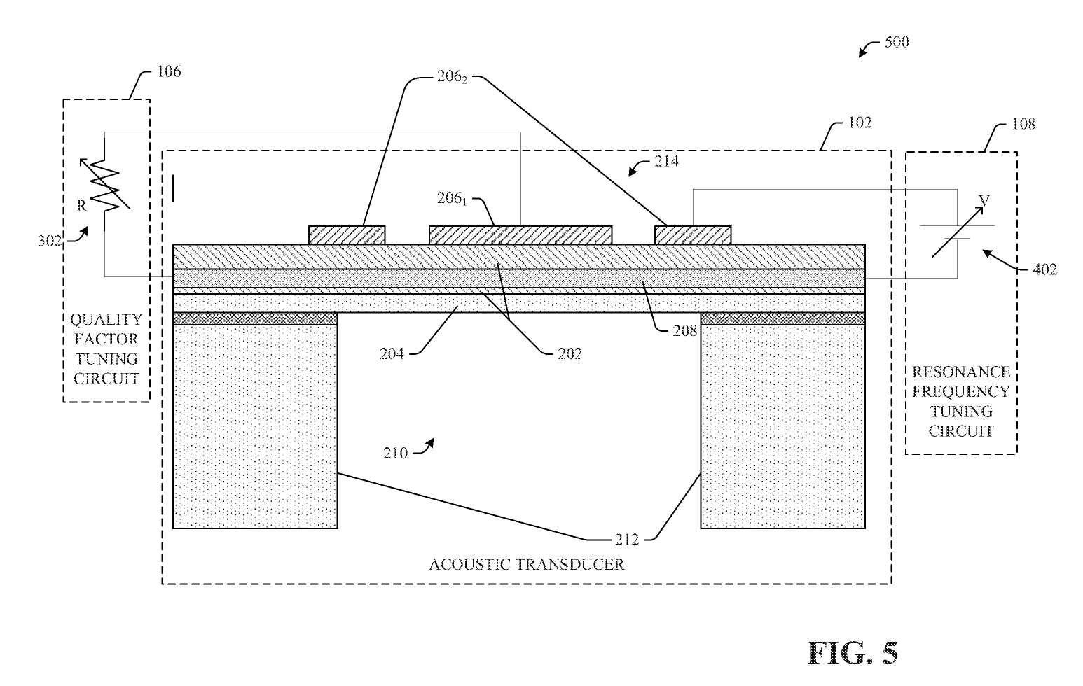

Referring now to FIG. 5, there illustrated is an example system 500 that facilitates electrically tuning both the quality factor and resonant frequency of the acoustic transducer 102. In this example system, the electrode layer can be split into quality factor tuning electrode 206₁ and frequency tuning electrode 206₂. The quality factor tuning electrode 206₁ can be coupled to the quality factor tuning circuit 106 and the frequency tuning 206₂ can be coupled to the resonance frequency tuning circuit 108. The operation of the quality factor tuning circuit 106 and the resonance frequency tuning circuit 108 is the same as (or substantially similar to) that described, more fully herein with respect to systems 300 and 400 respectively. It is noted that the acoustic transducer 102, the quality factor tuning circuit 106, the piezoelectric material 202, the supporting layer 204, the electrodes 206₂ and 208, the cavity 210, the substrate 212, the acoustic media 214, the resistor 302, and the voltage source 402 can operate and/or include functionality, as more fully described with respect to systems 100-400. Further, it is noted that the acoustic transducer 102 can comprise fewer of greater number of layers than those depicted in FIGS. 2-5.

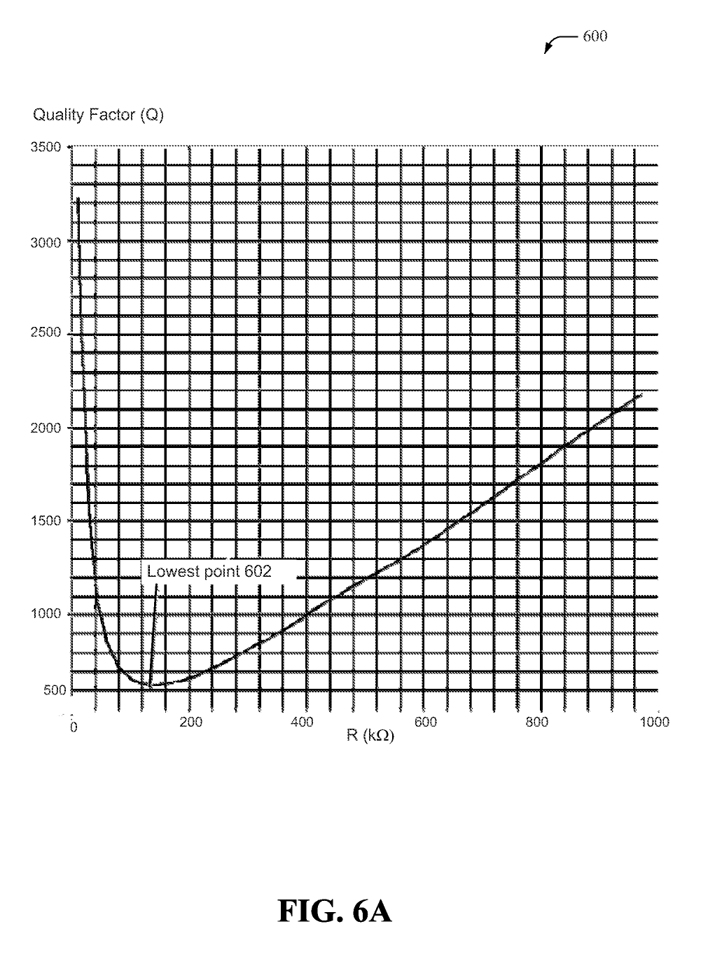

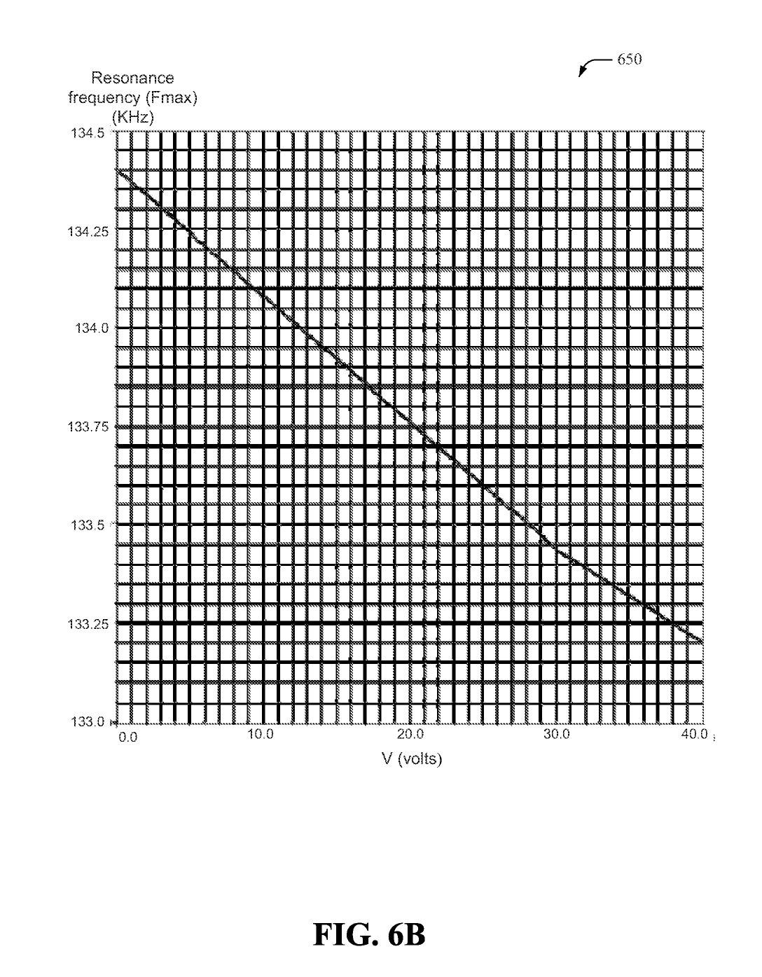

FIGS. 6A-6B illustrate simulation results for electrical tuning of parameters of the acoustic transducer 102, according to an aspect of the specification. As an example, a MEMS wafer with diameter D=800 um having a first top electrode 206₁ having diameter 0.8D and a second top electrode 206₂ placed 0.5D from the periphery can be utilized. Further, a pressure load of 1 Pascal (alternating current) is applied to the acoustic transducer 102. It is noted that the subject specification is not limited to these dimensions and/or pressure loads and most any dimensions and/or pressure loads can be utilized to form and/or applied to the acoustic transducer 102. Referring now to FIG. 6A, there depicted is an example graph 600 illustrating tuning of the quality factor of the acoustic transducer 102 based on modifying a resistance value R of the resistor 302. In one aspect, graph 600 can depict resistance R and quality factor Q observed in systems 300 and/or 500 (e.g., wherein V=0V). As an example, the resistance R can be changed from 10 kΩ to 1MΩ. As seen from 600, as the resistance R is varied (between 10 kΩ and 1MΩ), the quality factor (Q) changes between 625 and 3200. Moreover, initially, as the resistance is increased (to approximately 130 kΩ), the quality factor decreases, until it reaches a lowest point 602 at Q=625. The lowest point 602 indicates an impedance matched condition, wherein energy loss relative to the stored energy of the transducer is the fastest (e.g., energy transfer is most efficient).

With respect to FIG. 6B, there depicted is an example graph 650 illustrating tuning of the resonance frequency of the acoustic transducer 102 based on modifying a voltage value V of the DC voltage source 402. In one aspect, graph 600 can depict voltage V and resonance frequency Fmax observed in systems 400 and/or 500 (e.g., wherein R=1MΩ). As an example, the voltage V can be changed from 0V to 40V. As seen from 650, the resonance frequency Fmax is inversely proportional to the voltage V and as the voltage V is increased (from 0V to 40V), the resonance frequency Fmax decreases from 134.28 kilohertz (KHz) to 133.24 KHz. Accordingly, a 0.9% resonance frequency tuning can be implemented under bias voltage of 40V.

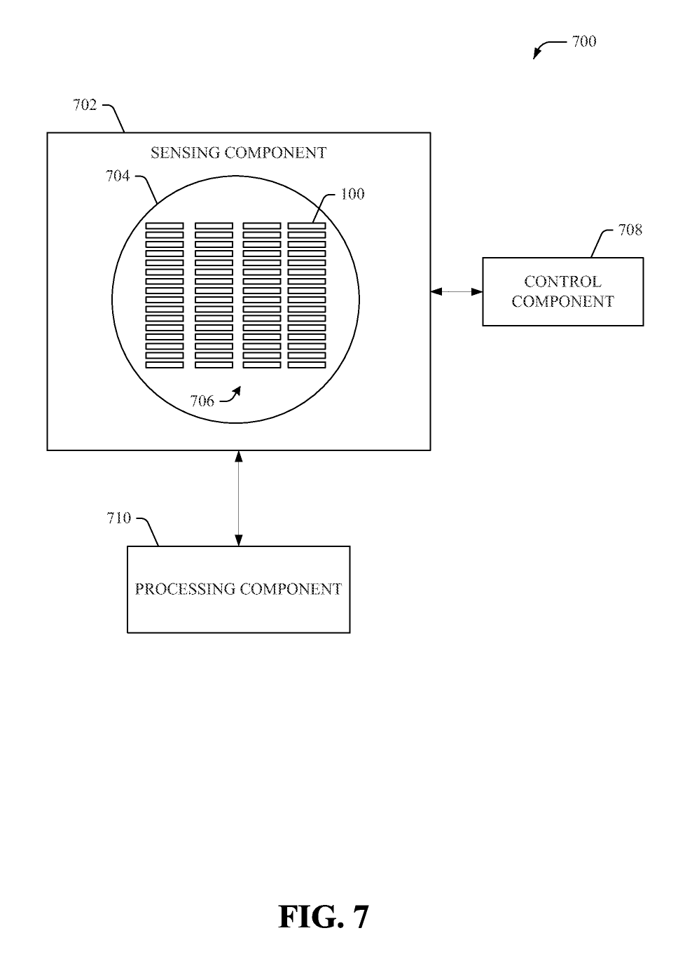

FIG. 7 illustrates an example system 700 utilized for analysis of acoustically sensed data in accordance with an aspect of the subject disclosure. System 700 can be utilized in various applications, such as, but not limited to, medical applications, security systems, biometric recognition systems, mobile communication systems, industrial automation systems, automotive systems, robotics, etc. In one aspect, system 700 can include a sensing component 702 that can facilitate acoustic sensing. Moreover, the sensing component 702 can include a silicon wafer 704 having a two-dimensional (or one-dimensional) array 706 of acoustic elements, for example, system 100 comprising the acoustic transducer 102 and the tuning component 104. As an example, the acoustic transducer 102 can comprise a pMUT device that, due to its smaller size, volume, weight, and/or power consumption, can be utilized in applications, such as, but not limited to medical imaging and/or mobile communication devices (e.g., biometric recognition systems, such as, but not limited to, fingerprint sensors and/or motion/gesture recognition sensors).

According to an aspect, a control component 708 can be utilized to control the values of the resistor 302 and/or the voltage source 402 to tune the quality factor and/or resonance frequency, respectively. In an example, the control component 708 can comprise most any circuitry or components that can determine and/or program values of resistor 302 and/or the voltage source 402 during a particular time period. For instance, the control component 708 can set the resistance of resistor 302 and/or the bias voltage of the voltage source 402 based on defined user/manufacturer policies, operating modes of the system 700, detection of events, etc.

During transmission, a set of transducers of the two-dimensional array 706 can transmit an acoustic signal (e.g., a short ultrasonic pulse) and during sensing, the set of transducers of the two-dimensional array 706 can detect an interference of the acoustic signal with an object (in the path of the acoustic wave). The received interference signal (e.g., generated based on reflections, echoes, etc. of the acoustic signal from the object) can then be analyzed by a processing component 710. As an example, the processing component 710 can determine an image of the object, a distance of the object from the sensing component 702, a density of the object, a motion of the object, etc., based on comparing a frequency and/or phase of the interference signal with a frequency and/or phase of the acoustic signal. Moreover, results generated by the processing component 710 can be further analyzed or presented to a user via a display device (not shown).

In one example, for fingerprinting applications, the object can be a human finger and the processing component 710 can determine, based on a difference in interference of the acoustic signal with valleys and ridges of the finger, an image depicting dermal and/or sub-dermal layers of the finger. Further, the processing component 710 can compare the image with a database of known fingerprint images to facilitate recognition and/or identification. In another example, for gesture/motion applications, the processing component 710 can determine, based on a difference in interference of the acoustic signal with an object (e.g., human hand), motion or gestures performed by the object. Further, the processing component 710 can compare the motion/gesture data with a database of known motions/gestures to determine a command that is to be performed (e.g., a hand wave can indicate that a device is to be switched on, a motion of a person can be utilized to move a pointer and/or avatar on a display screen, etc.). It is noted that the control component 708 and/or the processing component 710 can include one or more processors configured to confer at least in part the functionality of system 700. To that end, the one or more processors can execute code instructions stored in memory, for example, volatile memory and/or nonvolatile memory. By way of illustration, and not limitation, nonvolatile memory can include read only memory (ROM), programmable ROM (PROM), electrically programmable ROM (EPROM), electrically erasable PROM (EEPROM), or flash memory. Volatile memory can include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). The memory (e.g., data stores, databases) of the subject systems and methods is intended to comprise, without being limited to, these and any other suitable types of memory.

As it employed in the subject specification, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors can exploit nano-scale architectures such as, but not limited to, molecular and quantum-dot based transistors, switches and gates, in order to optimize space usage or enhance performance of user equipment. A processor may also be implemented as a combination of computing processing units.

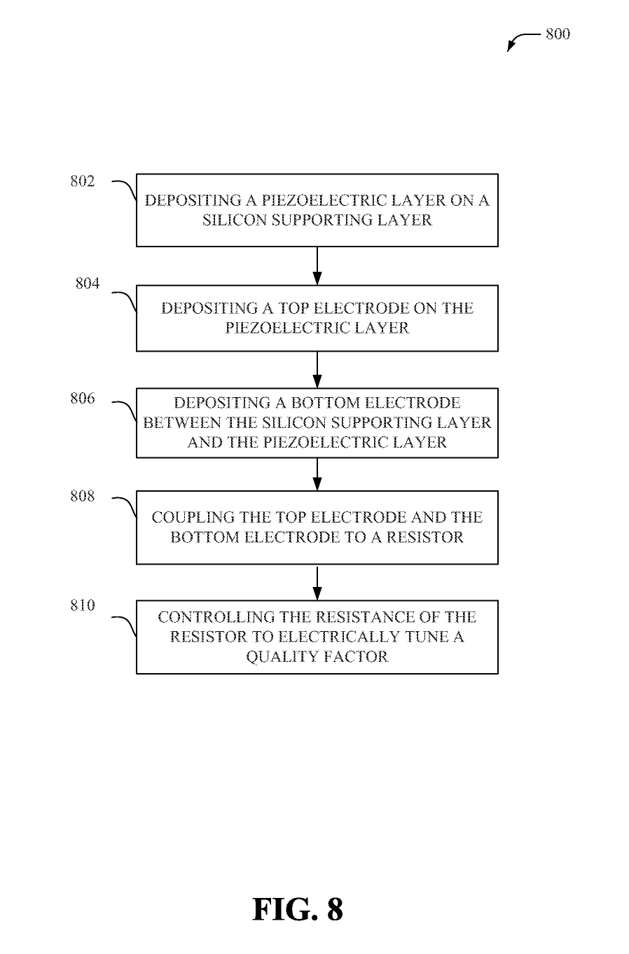

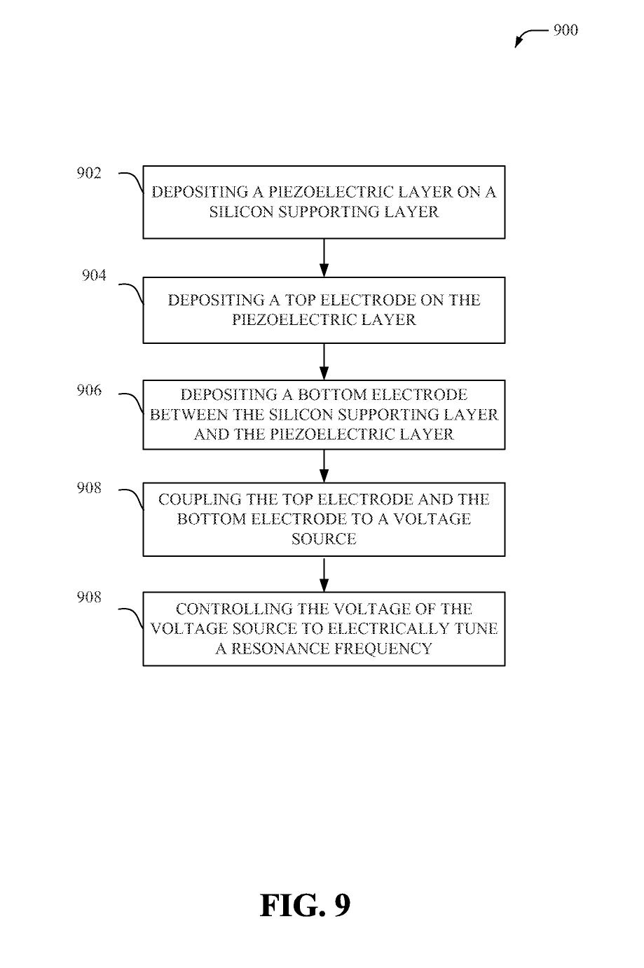

FIGS. 8-9 illustrate methodologies and/or flow diagrams in accordance with the disclosed subject matter. For simplicity of explanation, the methodologies are depicted and described as a series of acts. It is to be understood and appreciated that the subject innovation is not limited by the acts illustrated and/or by the order of acts, for example acts can occur in various orders and/or concurrently, and with other acts not presented and described herein. Furthermore, not all illustrated acts may be required to implement the methodologies in accordance with the disclosed subject matter. In addition, the methodologies could alternatively be represented as a series of interrelated states via a state diagram or events. Additionally, it should be further appreciated that the methodologies disclosed hereinafter and throughout this specification are capable of being stored on an article of manufacture to facilitate transporting and transferring such methodologies to computers. The term article of manufacture, as used herein, is intended to encompass a computer program accessible from any computer-readable device or computer-readable storage/communications media.

FIG. 8 illustrates an example methodology 800 for controlling a quality factor of an acoustic sensor in accordance with an aspect of the subject disclosure. Specifically, methodology 800 employs a path for dissipation of energy that is stored in the acoustic sensor due to the damping. At 802, a piezoelectric layer can be deposited on a silicon supporting layer. The piezoelectric layer converts acoustic energy into electrical energy and vice versa. As an example, MN can be utilized for the piezoelectric layer and SiO₂ can be utilized as the silicon supporting layer. At 804, a top electrode can be deposited on the piezoelectric layer and at 806, a bottom electrode can be deposited between the silicon supporting layer and the piezoelectric layer. As an example, metals, such as, but not limited to Al/Ti, Mo, etc. can be patterned to form electrodes in different shapes (e.g., circle, square, octagon, hexagon, etc.) that are defined in-plane with the sensor membrane. Further, the top electrode can be located at an area of the piezoelectric layer that experiences maximum strain/deformation.

In one aspect, at 808, the top electrode and the bottom electrode can be coupled to a resistor. As an example, the resistor can have a variable resistance, e.g., 10 kΩ-1MΩ and can be programmable (e.g., resistance value can change based on defined instructions/policies, events, operating modes, etc.) and/or time variant (e.g., resistance value can change based on time). At 810, the resistance of the resistor is controlled to electrically tune the quality factor of the acoustic sensor. According to an aspect, the resistor provides an energy dissipation path (e.g., heat) alternative to mechanical damping. Moreover, the resistor can be a component, circuitry or combination thereof that can perform like an energy dissipation device.

FIG. 9 illustrates an example methodology 900 for controlling a resonance frequency of an acoustic sensor in accordance with an aspect of the subject disclosure. In one aspect, methodology 900 introduces tensile or compressive stress in the piezoelectric layer of the acoustic sensor to compensate for a resonance frequency drift. At 902, a piezoelectric layer can be deposited on a silicon supporting layer. The piezoelectric layer converts acoustic energy into electrical energy and vice versa. As an example, MN can be utilized for the piezoelectric layer and SiO₂ can be utilized as the silicon supporting layer. At 904, a top electrode can be deposited on the piezoelectric layer and at 906, a bottom electrode can be deposited between the silicon supporting layer and the piezoelectric layer. As an example, metals, such as, but not limited to Al/Ti, Mo, etc. can be patterned to form electrodes in different shapes (e.g., circle, square, octagon, hexagon, etc.) that are defined in-plane with the sensor membrane. Further, the top electrode can be located at an area that is close to (e.g., within a defined distance from) a constraint (e.g., a point at which the membrane can be easily strengthened) of the piezoelectric layer.

In one aspect, at 908, the top electrode and the bottom electrode can be coupled to a voltage source, for example a DC voltage source. As an example, the voltage source can have a variable bias voltage, e.g., 0V-40V and can be programmable (e.g., bias voltage value can change based on defined instructions/policies, events, operating modes, etc.) and/or time variant (e.g., bias voltage value can change based on time). At 910, the bias voltage of the voltage source is controlled to electrically tune the resonance frequency of the acoustic sensor. According to an aspect, the bias voltage generates tensile or compressive stress in the piezoelectric layer which stiffens or softens the equivalent spring constant of the piezoelectric layer. As the equivalent spring constant changes, the resonance frequency is modified. Moreover, as the bias voltage is increased, the resonance frequency is decreased and as the bias voltage is decreased, the resonance frequency is increased.

What has been described above includes examples of the subject disclosure. It is, of course, not possible to describe every conceivable combination of components or methodologies for purposes of describing the subject matter, but it is to be appreciated that many further combinations and permutations of the subject disclosure are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. Further, the components and circuitry elements described above can be of any suitable value in order to implement the embodiments of the present invention. For example, the resistors can be of any suitable resistance, voltage sources can provide any suitable voltages, etc.

The aforementioned systems/circuits/components have been described with respect to interaction between several components. It can be appreciated that such systems/circuits and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described herein may also interact with one or more other components not specifically described herein.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A piezoelectric actuated transducer, comprising:
a piezoelectric layer deposited on a silicon supporting layer;
a first electrode layer deposited on the piezoelectric layer; and
a second electrode layer deposited between the silicon supporting layer and the piezoelectric layer, wherein at least a portion of the first electrode layer and the second electrode layer are coupled to a direct current voltage source to facilitate electrical tuning of a resonant frequency of the piezoelectric actuated transducer.

2. The piezoelectric actuated transducer of claim 1, wherein the second electrode layer comprises a supporting silicon layer having a doping level that exceeds a defined threshold.

3. The piezoelectric actuated transducer of claim 1, wherein a voltage of the direct current voltage source is programmable.

4. The piezoelectric actuated transducer of claim 1, wherein a voltage of the direct current voltage source is time variant.

5. The piezoelectric actuated transducer of claim 4, wherein the voltage is modified based on a mode of operation of the piezoelectric actuated transducer.

6. The piezoelectric actuated transducer of claim 1, further comprising:
a released structure comprising the silicon supporting layer, wherein a shape of the released structure is defined in-plane with a microelectromechanical systems (MEMS) membrane that comprises the piezoelectric layer, the first electrode layer and the second electrode layer.

7. The piezoelectric actuated transducer of claim 6, wherein the released structure operates in at least one of vacuum, air, or a liquid.

8. The piezoelectric actuated transducer of claim 1, wherein a shape of at least the portion of the first electrode layer and the second electrode layer is defined in-plane with a microelectromechanical systems (MEMS) membrane that comprises the piezoelectric layer, the first electrode layer and the second electrode layer.

9. The piezoelectric actuated transducer of claim 1, wherein at least the portion of the first electrode layer and the second electrode layer are placed at a location associated with a constraint of a microelectromechanical systems (MEMS) membrane that comprises the piezoelectric layer, the first electrode layer and the second electrode layer, wherein the constraint is a flexible structure in connection to an anchor of the MEMS membrane.

10. The piezoelectric actuated transducer of claim 9, wherein the MEMS membrane is a circular MEMS membrane and the location is at a periphery of the circular MEMS membrane.

11. The piezoelectric actuated transducer of claim 1, wherein the portion is a first portion and at least a second portion of the first electrode layer and the second electrode layer are coupled to a variable resistor to facilitate electrical tuning of a quality factor of the piezoelectric actuated transducer.

12. A piezoelectric actuated transducer, comprising:
a piezoelectric layer deposited on a silicon supporting layer;
a first electrode layer deposited on the piezoelectric layer; and
a second electrode layer deposited between the silicon supporting layer and the piezoelectric layer, wherein at least a portion of the first electrode layer and the second electrode layer are coupled to a resistor to facilitate electrical tuning of a quality factor of the piezoelectric actuated transducer.

13. The piezoelectric actuated transducer of claim 12, wherein the second electrode layer comprises a supporting silicon layer having a doping level that exceeds a defined threshold.

14. The piezoelectric actuated transducer of claim 12, wherein a resistance of the resistor is programmable.

15. The piezoelectric actuated transducer of claim 12, wherein a resistance of the resistor is time variant.

16. The piezoelectric actuated transducer of claim 15, wherein the resistance is modified based on a mode of operation of the piezoelectric actuated transducer.

17. The piezoelectric actuated transducer of claim 12, further comprising:
a released structure comprising the silicon supporting layer, wherein a shape of the released structure is defined in-plane with a microelectromechanical systems (MEMS) membrane that comprises the piezoelectric layer, the first electrode layer and the second electrode layer.

18. The piezoelectric actuated transducer of claim 17, wherein the released structure operates in at least one of vacuum, air, or a liquid.

19. The piezoelectric actuated transducer of claim 12, wherein a shape of at least the portion of the first electrode layer and the second electrode layer is defined in-plane with a microelectromechanical systems (MEMS) membrane that comprises the piezoelectric layer, the first electrode layer and the second electrode layer.

20. The piezoelectric actuated transducer of claim 12, wherein the first electrode layer and the second electrode layer are placed at a defined area of a microelectromechanical systems (MEMS) membrane that comprises the piezoelectric layer, the first electrode layer and the second electrode layer, wherein the defined area satisfies a specified strain criterion.

21. The piezoelectric actuated transducer of claim 20, wherein the MEMS membrane is a circular MEMS membrane and the defined area is at a center of the circular MEMS membrane.

22. The piezoelectric actuated transducer of claim 12, wherein the first electrode layer and the second electrode layer are coupled to a voltage source to facilitate electrical tuning of a resonant frequency of the piezoelectric actuated transducer.

23. A method, comprising:
depositing a piezoelectric layer on a silicon supporting layer;
forming a first electrode layer on the piezoelectric layer and a second electrode layer between the silicon supporting layer and the piezoelectric layer to form a piezoelectric actuated transducer; and
tuning a resonant frequency of the piezoelectric actuated transducer based on controlling a voltage signal across at least a portion of the first electrode layer and the second electrode layer.

24. The method of claim 23, wherein the forming the second electrode layer comprises doping a supporting silicon layer, wherein a doping level of the supporting silicon layer exceeds a defined threshold.

25. The method of claim 23, wherein the controlling the voltage signal comprises controlling the voltage signal based on a mode of operation of the piezoelectric actuated transducer.

26. The method of claim 23, further comprising:
forming a released structure comprising the silicon supporting layer, the piezoelectric layer, the first electrode layer and the second electrode layer; and
operating the released structure in at least one of vacuum, air, or a liquid.

27. The method of claim 23, wherein the forming the first electrode layer and the second electrode layer comprises forming at least the portion of the first electrode layer and the second electrode layer at a location associated with a constraint of a microelectromechanical systems (MEMS) membrane that comprises the piezoelectric layer, the first electrode layer and the second electrode layer, wherein the constraint is a flexible structure in connection to an anchor of the MEMS membrane.

28. The method of claim 23, wherein the portion is a first portion and the method further comprises:
tuning a quality factor of the piezoelectric actuated transducer based on controlling a resistance across at least a second portion of the first electrode layer and the second electrode layer.

29. The method of claim 28, wherein the forming the first electrode layer and the second electrode layer comprises forming at least the second portion of the first electrode layer and the second electrode layer at a location of a microelectromechanical systems (MEMS) membrane that comprises the piezoelectric layer, the first electrode layer and the second electrode layer, wherein the location is determined to satisfy a defined strain criterion.

30. A biometric sensing method, comprising:
transmitting an ultrasonic signal by an acoustic sensing element having a voltage source coupled across a first electrode that is deposited on a piezoelectric layer and a second electrode that is deposited below the piezoelectric layer;
sensing, by the piezoelectric layer, an interference signal that is generated based on an interference of the ultrasonic signal with an object; and
controlling a voltage of the voltage source to tune a resonant frequency of the piezoelectric layer.

31. The biometric sensing method of claim 30, comprising a fingerprint sensing method, wherein the object comprises a finger.
